(19) United States
(12) Patent Application Publication
(10) Pub. No.: US 2010/0099650 A1
(43) Pub. Date: Apr. 22, 2010

(54) NASAL SPRAY COMPOSITION AND METHOD FOR TREATING RHINITIS, SINUSITIS OR BOTH

(76) Inventor: Kent A. Knauer, Novelty, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/445,113

(22) PCT Filed: Oct. 5, 2007
(86) PCT No.: PCT/US07/80530

§ 371 Date: Apr. 10, 2009

Related U.S. Application Data
(60) Provisional application No. 60/850,560, filed on Oct. 10, 2006.

(51) Int. Cl. A61K 31/56; A61K 31/4164; A61P 29/00

(52) U.S. Cl. 514/171; 514/396

(57) ABSTRACT

A nasal spray composition for treating mucosal inflammation associated with rhinitis, sinusitis, or both can include a decongestant and at least one therapeutic agent. The therapeutic agent is selected from the group consisting of an anti-inflammatory agent and an anti-histamine agent. The nasal spray composition is non-habituating and is administered intranasally to a subject in need thereof.

### RELATED APPLICATION

This application claims priority from U.S. Provisional Application No. 60/850,560, filed Oct. 10, 2006, the subject matter which is incorporated herein by reference.

### TECHNICAL FIELD

The present invention generally relates to a composition and method for treating mucosal inflammation, and more particularly to a non-habituating nasal spray composition and method for intranasally treating rhinitis, sinusitis, or both.

### BACKGROUND OF THE INVENTION

Rhinitis, an inflammation of the nasal mucosal membrane, is characterized by sneezing, rhinorrhea, nasal congestion, and increased nasal secretion. When these conditions persist for a period of more than three weeks, they are termed “chronic.” More than 37 million Americans, particularly those with allergies or asthma, suffer from these conditions, making them the most common chronic medical problems in the United States. Failure to effectively treatment rhinitis may lead to other disorders including infection of the ears, lower respiratory tract, and sinuses.

Inflammation of the sinuses, known as rhinosinusitis or sinusitis, is difficult to treat successfully. In general, treatment consists of a combination of antibiotics and decongestants or antihistamines. In addition, steroid nasal sprays are commonly used to reduce inflammation. For patients with severe chronic sinusitis, oral steroids such as prednisone may also be prescribed. Oral steroids, however, often have significant side effects, and the long-term safety of steroid administration, especially in children, is not fully understood. When drug therapy fails, surgery is usually the only alternative.

Administration of nasal sprays to the nasal mucosa requires delivery of a precise dosage and adherence to a strict regimen as prescribed by a physician or as detailed by the packaging instructions of an over-the-counter medicine. Often, patients may not follow the instructions and presume that taking a larger than directed dose (“over-medication” or “over-dosage”) will provide a speedy recovery. While the over-medication may temporarily improve the congestion, side effects of over-medication or prolonged use can include addiction to the decongestant compositions, significant “rebounding” (swelling-relaxing-swelling patterns known as Rhinitis medicamentosa), and may lead to burning, itching, and drying of the nasal passage.

### SUMMARY OF THE INVENTION

According to one aspect of the present invention, a nasal spray composition for treating mucosal inflammation associated with rhinitis and/or sinusitis can include a decongestant and at least one therapeutic agent. The therapeutic agent may be selected from the group consisting of an anti-inflammatory agent and an anti-histamine agent. The nasal spray composition is non-habituating and can be administered intranasally to a subject in need thereof.

According to another aspect of the present invention, a method is provided for treating rhinitis and/or sinusitis in a subject. The method can include intranasally administering a therapeutically effective amount of a non-habituating nasal spray composition to the subject. The nasal spray composition can include a decongestant and at least one therapeutic agent selected from the group consisting of an anti-inflammatory agent and an anti-histamine agent.

### BRIEF DESCRIPTION OF THE DRAWINGS

The foregoing and other features of the present invention will become apparent to those skilled in the art to which the present invention relates upon reading the following description with reference to the accompanying drawings, in which:

FIGS. 1A-D are a series of CT images showing the effect of a nasal spray composition according to the present invention on a subject with chronic sinusitis. FIG. 1A shows the persistence of inflammation and air fluid level on the right, as well as swelling of the nasal turbinates. After repeated antibiotic administration and the addition of the nasal spray composition, there was resolution of the R max sinus and significant reduction in swelling of the nasal turbinates (FIG. 1B); and

FIGS. 2A-B are a series of CT images showing the effect of the nasal spray composition on a subject with sinusitis. FIG. 2A shows an opacified R max sinus before treatment with the nasal spray composition. FIG. 2B shows complete resolution and clearing of the R osteomeatal complex area after three weeks of treatment with the nasal spray composition.

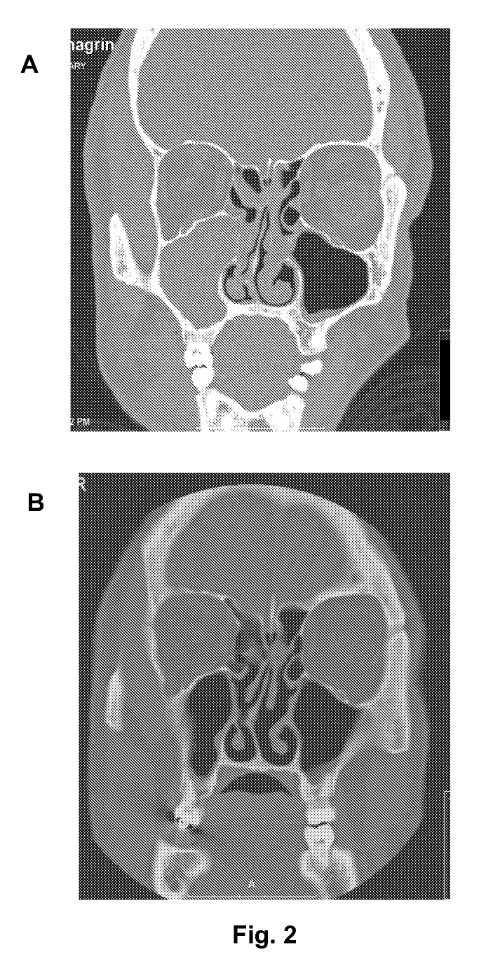

### DETAILED DESCRIPTION

The present invention generally relates to a pharmaceutical composition and method for treating mucosal inflammation, and more particularly to a non-habituating nasal spray composition and method for intranasally treating rhinitis and/or sinusitis. The present invention is based on the discovery that a nasal spray composition comprising a decongestant and at least one therapeutic agent can effectively reduce or eliminate symptoms associated with inflammation of the nasal mucosa, i.e., rhinitis, sinusitis, or both. More particularly, the present invention is based on the discovery that intranasal use of the nasal spray composition is not habit forming or addictive when used to treat symptoms associated with rhinitis, sinusitis, or both. Based on this discovery, the present invention provides a nasal spray composition and method for treating mucosal inflammation associated with rhinitis and/or sinusitis.

All scientific and technical terms used in this application have meanings commonly used in the art unless otherwise specified. The definitions provided herein are to facilitate understanding of certain terms used frequently herein and are not meant to limit the scope of the present invention.

In the context of the present invention, the terms “treat,” “therapy,” and the like mean alleviating, slowing the progression, preventing, attenuating, or curing mucosal inflammation associated with rhinitis, sinusitis, or both.

As used herein, the term “decongestant” refers to any agent or ingredient for reducing or eliminating congestion of the nasal passages by widening the passages, stimulating the release of phlegm and mucus from these passages, and/or reducing the swelling of the mucous membranes in the passages.

As used herein, the term “anti-inflammatory agent” refers to any compound or ingredient that acts against, counters, decreases, diminishes, inhibits, or reduces inflammation or an inflammatory response. “Inflammation” refers to a response to infection and/or injury in which cells involved in detoxification and repair are mobilized to a compromised site by inflammatory mediators. Examples of the inflammatory response can include increased mucus production, edema, vasodilation, fever and pain.

As used herein, the term “non-steroidal anti-inflammatory drug or NSAID” refers to any non-narcotic analgesic/non-steroidal anti-inflammatory compound selected from the group consisting of chromones, propionic acid derivatives, acetic acid derivatives, fenamic acid derivatives, biphenylcarboxylic acid derivatives, Cox-2 inhibitors and oxicams.

As used herein, the term “corticosteroid” refers to a class of compounds useful in treatment of inflammatory conditions, including those resulting from infection. Corticosteroids can include compounds that are naturally occurring, synthetic, or semi-synthetic in origin, and are characterized by the presence of a steroid nucleus of four fused ring structures.

As used herein, the term “anti-histamine agent” refers to any of various compounds that can counteract histamine in the body, and that may be used for treating allergic reactions and/or cold symptoms.

As used herein, the term “rhinitis” refers to inflammation of the nasal mucous membranes resulting from, e.g., a cold, flu, or allergies. Rhinitis may be characterized by one or more cold-like symptoms including, for example, rhinorrhea, sneezing, nasal congestion, and increased nasal secretion. Rhinitis can include acute rhinitis, chronic rhinitis, allergic rhinitis, seasonal allergic rhinitis, perennial allergic rhinitis, vasomotor rhinitis, infectious rhinitis, and atrophic rhinitis.

As used herein, the term “sinusitis” refers to inflammation of the paranasal sinuses, which can be the result of infection (e.g., bacterial, fungal or viral), allergic or autoimmune causes. It should be appreciated that newer classifications of sinusitis may refer to the condition as “rhinosinusitis” since inflammation of the sinuses typically does not occur without some inflammation of the nose as well.

As used herein, the term “therapeutically effective amount” refers to an amount sufficient to elicit a desired biological response. The desired biological response can include a reduction (complete or partial) of at least one symptom associated with rhinitis, sinusitis, or both. For example, a reduction in nasal mucous production may be considered a desired biological response.

As used herein, the term “subject” refers to a mammal undergoing treatment for mucosal inflammation associated with rhinitis, sinusitis, or both. Mammals can include mice, rats, cats, guinea pigs, hamsters, dogs, horses, cows, monkeys, chimpanzees and humans.

Very few drugs relieve a symptom as effectively as an over-the-counter decongestant nasal spray relieves a stuffy nose. With some nasal sprays, a single dose can relieve symptoms for as long as 12 hours. But relief provided by nasal spray decongestants can come at a price: the risk of rebound congestion caused by overuse and, for some people, a vicious cycle of overuse and dependence akin to an addiction (Snow, SS et al., Br. J. Psychiatry 136:297-299 (1980); Graf, P and Juto, J E Rhinology 33(1):14-17 (1995)). Rhinitis medicamentosa (RM), which is a condition of rebound nasal congestion, can be brought on by extended use of topical decongestants (e.g., oxymetazoline, phenylephrine, and xylometazoline nasal sprays). This condition typically occurs after 5 to 7 days of use of such medications. Patients often try increasing both the dose and the frequency of nasal sprays upon the onset of RM, in turn worsening the condition (Lin C Y et al., Ann. Owl. Rhinol. Laryngol. 113(2):147-51 (2004)).

In one aspect of the present invention, a nasal spray composition whose use does not promote or cause habituation, dependence, and/or addiction is provided. The nasal spray composition can comprise a decongestant and at least one therapeutic agent selected from the group consisting of an anti-inflammatory agent and an anti-histamine agent. As described in more detail below, the nasal spray composition can be used in a lesser amount, and with greater efficacy, as compared to known nasal spray compositions. By providing a non-habituating, fast-acting, and efficacious nasal spray composition, the present invention may be useful for preventing or reducing RM while also reducing or eliminating mucosal inflammation associated with rhinitis, sinusitis, or both.

The decongestant can comprise any agent or ingredient that actively reduces or eliminates congestion of the nasal passages by, for example, widening the nasal passages and/or by stimulating the release of phlegm or mucus from the passages. The decongestant can comprise about 0.1% to about 50% or greater by weight of the nasal spray composition. More particularly, a decongestant such as oxymetazoline hydrochloride may comprise about 0.1% to about 50% or greater by weight of the nasal spray composition.

Other decongestants can include, without limitation, naphazoline hydrochloride, phenylethylamine, dopamine, dobutamine, colterol, ethylnorepinephrine, isoproterenol, isoetharine, metaproterenol, terbutaline, metaraminol, tyraine, hydroxyamphetamine, ritodrine, prenalterol, methoxyamine, albuterol, amphetamine, methamphetamine, benzphetamine, mephentermine, phentermine, fenfluramine, propylhexedrine, diethylpropion, phenmetrazine, phendimetrazine, xyloepinephrine, ephedrine, pseudoephedrine, phenylephedrine, phenylpropanolamine, phenylephrine hydrochloride, xylometaxoline hydrochloride, and pharmaceutically acceptable salts and mixtures thereof.

In another aspect of the present invention, the anti-inflammatory agent can comprise any agent or ingredient that acts against, counters, decreases, diminishes, inhibits, or reduces inflammation or an inflammatory response. For instance, an anti-inflammatory agent can include an NSAID or a corticosteroid. An NSAID may include any non-narcotic analgesic/non-steroidal anti-inflammatory compound within one of six chemical classes of compounds including, but not limited to, chromones, propionic acid derivatives, acetic acid derivatives, fenamic acid derivatives, biphenylcarboxylic acid derivatives, Cox-2 inhibitors and oxicams.

Propionic acid derivatives can include, but are not limited to, ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, and bucloxic acid. Structurally related propionic acid derivatives having similar analgesic and/or anti-inflammatory properties may also be included.

Acetic acid derivatives can include, but are not limited to, indomethacin, sulindac, tolmetin, zomepirac, diclofenac, fenchlofenac, alchlofenac, ibufenac, isoxepac, furofenac, tiopinac, zidometacin, acemetacin, fentiazac, clidanac and oxipinac. Structurally related acetic acid derivatives having similar analgesic and/or anti-inflammatory properties may also be included.

Fenamic acid derivatives can include, but are not limited to, mefenamic acid, meclofenamic acid, flufenamic acid, niflumic acid and tolfenamic acid. Structurally related fenamic acid derivatives having similar analgesic and/or anti-inflammatory properties may also be included.

Biphenylcarboxylic acid derivatives can include, but are not limited to, diflunisal and flufenisal. Structurally related biphenylcarboxylic acid derivatives having similar analgesic and/or anti-inflammatory properties may also be included.

Oxicams can include, but are not limited to, piroxicam, sudoxicam, isoxicam. Structurally related oxicams having similar analgesic and/or anti-inflammatory properties may also be included.

Cox-2 inhibitors can include compounds which selectively inhibit Cox-2 over Cox-1. Examples of Cox-2 inhibitors include, without limitation, rofecoxib, etoricoxib, valdecoxib, parecoxib, lumiracoxib, tiracoxib, ABT963, CS502 and GW406381.

In one particular aspect of the present invention, the nasal spray composition can comprise a decongestant and an NSAID. The NSAID may comprise about 0.1% to about 50% or greater by weight of the nasal spray composition, and the decongestant may comprise about 0.1% to about 50% or greater by weight of the nasal spray composition. More particularly, the nasal spray composition can comprise about 0.1% to about 50% or greater by weight of oxymetazoline hydrochloride, and about 0.1% to about 50% or greater by weight of a chromone, such as cromolyn sodium.

In another aspect of the present invention, the corticosteroid can include any member of a class of compounds useful in treatment of inflammatory conditions, including those resulting from infection. Corticosteroids may be generally characterized by the presence of a steroid nucleus of four fused ring structures. The corticosteroid can comprise about 0.1% to about 50% or greater by weight of the nasal spray composition. More particularly, the nasal spray composition can comprise about 0.1% to about 50% or greater by weight of oxymetazoline hydrochloride, and about 0.1% to about 50% or greater by weight of a corticosteroid, such as triamcinolone acetonide.

Other corticosteroids can include, without limitation, hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, mometasone furoate, ciclesonide, loteprednol, fluticasone propionate, flunisolide, budesonide, and pharmaceutically acceptable salts and mixtures thereof.

In one example of the present invention, the nasal spray composition can include a corticosteroid, such as NASACORT AQ, and a decongestant, such as AFRIN. The nasal decongestant can be formed by obtaining a desired amount of AFRIN (e.g., about 1 oz.) and then combining the desired amount of AFRIN with a desired amount of NASACORT AQ. For example, four sample bottles (e.g., about 6.5 g each) or one prescription bottle (e.g., about 16.5 g) of NASACORT AQ can be mixed with the desired amount of AFRIN in a single container or device capable of delivering the NASACORT AQ/AFRIN mixture intranasally.

In another aspect of the present invention, the nasal spray composition can comprise a decongestant and an anti-histamine agent. The anti-histamine agent can comprise any compound capable of counteracting histamine in a subject, and may be useful for treating allergic reactions and/or cold symptoms. The anti-histamine agent can comprise about 0.1% to about 50% or greater by weight of the nasal spray composition. More particularly, the nasal spray composition may comprise about 0.1% to about 50% or greater by weight of oxymetazoline hydrochloride, and about 0.1% to about 50% or greater by weight of an anti-histamine agent, such as azelastine.

Other antihistamine agents can include, without limitation, brompheniramine maleate, chlorpheniramine maleate, doxylamine succinate, phenindamine tartate, pheniramine maleate, promethazine maleate, pyrilamine maleate, thonzylamine hydrochloride, astemizole, azatadine, acrivastine, cetirizine, clemastine, cyclizine, carebastine, cyproheptadine, carbinoxanine, descarboethoxyloratadine, desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine, triprolidine, diphenhydramine, oxatomide, setastine, tazifyline, phenyltoloxamine, and pharmaceutically acceptable salts and mixtures thereof.

It should be appreciated that the nasal spray composition may comprise combinations of decongestants and therapeutic agents other than those described above. For example, the nasal spray composition can include a decongestant, an NSAID, and a corticosteroid. Alternatively, the nasal spray composition can include a decongestant, a corticosteroid, and an anti-histamine agent. Further, the nasal spray composition can include a decongestant, an NSAID, a corticosteroid, and an anti-histamine agent.

In another aspect of the present invention, the nasal spray composition may additionally comprise a pharmaceutically acceptable carrier, such as a diluent, to facilitate delivery of the nasal spray composition. For example, where delivery of the nasal spray composition in a powder form is desired, the pharmaceutically acceptable carrier can comprise a suitable powder base such as talc, lactose starch, or the like.

Alternatively, where delivery of the nasal spray composition in a droplet or spray form is desired, the pharmaceutically acceptable carrier can comprise an aqueous carrier such as saline, for example. Aqueous carriers can contain about 0.1% to about 2.0% by weight of a salt, e.g., sodium chloride. The nasal composition can be isotonic, i.e., it has the same osmotic pressure as blood and lacrimal fluid. Suitable non-toxic pharmaceutically acceptable carriers are known to those skilled in the art.

The choice of a pharmaceutically acceptable carrier may depend upon the nature of the particular nasal dosage form required, e.g., whether the active agent(s) (i.e., a decongestant and a therapeutic agent) is formulated into a nasal solution (i.e., for use as drops or as a spray), a nasal suspension, a nasal ointment, a nasal gel or another nasal form. It should be appreciated that other ingredients such as pH adjusters (e.g., an acid such as HCl), emulsifiers or dispersing agents, buffering agents, preservatives, wetting agents, and gelling agents may also be included in the nasal spray composition.

Consistency aids may also be included in the nasal spray composition. Consistency aids can comprise low molecular weight mono- and polyols selected from the group consisting of monosaccharides (e.g., glucose and fructose), disaccharides (e.g., sucrose, lactose, maltose or cellobiose) and other sugars, ribose, glycerine, sorbitol, xylitol, inositol, propylene glycol, galactose, mannose, xylose, rhamnose, glutaraldehyde, invert sugars, ethanol, honey, mannitol, polyethylene glycol, glycerol, and mixtures thereof.

Consistency aids may provide enhanced physical stability and the proper consistency of the nasal spray composition prior to administration so that an optimal degree of spreading over the mucosa is achieved after administration. For example, consistency aids may reduce or delay the rate at which the active agents in the nasal spray composition are adsorbed by the mucin of the mucosa. This may permit the nasal spray composition to better spread and coat the nasal mucosa.

In another aspect of the present invention, a method is provided for treating mucosal inflammation associated with rhinitis, sinusitis or both. According to the present invention, rhinitis may generally include any inflammation of the nasal mucous membrane. Symptoms of rhinitis can generally include one or more cold-like symptoms including, for example, rhinorrhea, increased nasal secretion, nasal congestion, sneezing and catarrh. Rhinitis can also include both allergic rhinitis and non-allergic rhinitis. “Allergic rhinitis” refers to any allergic reaction of the nasal mucosa and may include hay fever (seasonal allergic rhinitis) and perennial rhinitis (non-seasonal allergic rhinitis). “Non-allergic rhinitis” refers to eosinophilic non-allergic rhinitis which is found in subjects with negative skin tests and those who have numerous eosinophils in their nasal secretions.

Also according to the present invention, sinusitis can include a condition that is similar to rhinitis generally characterized by inflammation of the paranasal sinuses. Sinusitis can be acute (i.e., less than four weeks), subacute (i.e., 4-12 weeks) or chronic (i.e., for 12 weeks or more), and can include such symptoms as headache, upper jaw and teeth pain, swelling of the eyelids and ocular tissue, and superficial pain associated with tactile compression of the nose.

For nasal administration of the nasal spray composition, various devices are available in the art for the generation of drops, droplets and sprays. For example, the nasal spray composition can be administrated into the nasal passages of a subject by means of a dropper (or pipet) that includes a glass, plastic or metal dispensing tube. Fine droplets and sprays can be provided by an intranasal pump dispenser or squeeze bottle as well known in the art.

Other means for delivering the nasal spray composition, such as inhalation via a metered dose inhaler (MDI), may also be used according to the present invention. Several types of MDIs are regularly used for administration by inhalation. These types of devices can include breath-actuated MDI, dry powder inhaler (DPI), spacer/holding chambers in combination with MDI, and nebulizers. The term “MDI” as used herein refers to an inhalation delivery system comprising, for example, a canister containing an active agent dissolved or suspended in a propellant optionally with one or more excipients, a metered dose valve, an actuator, and a mouthpiece. The canister is usually filled with a solution or suspension of an active agent, such as the nasal spray composition, and a propellant, such as one or more hydrofluoroalkanes. When the actuator is depressed a metered dose of the solution is aerosolized for inhalation. Particles comprising the active agent are propelled toward the mouthpiece where they may then be inhaled by a subject.

In an example of the method, a subject suffering from symptoms of chronic sinusitis, such as nasal congestion and inflammation of the paranasal sinuses, may be treated with a therapeutically effective amount of a nasal spray composition. The nasal spray composition can comprise about 0.1% to about 50% or greater by weight of a decongestant, such as AFRIN, and about 0.1% to about 50% or greater by weight of a corticosteroid, such as NASACORT AQ. The nasal spray composition can be prepared as described above, for example, by mixing about 1 oz. of AFRIN with four sample bottles (or one prescription bottle) of NASACORT AQ.

The nasal spray composition can additionally or optionally comprise a pharmaceutically acceptable carrier, such as a 0.1% to 2.0% saline solution, which may facilitate intranasal administration of the nasal spray composition. The nasal spray composition may be packaged in a squeeze bottle having a plastic dispensing tube so that the nasal spray composition can be sprayed as a fine mist into a nasal passage or passages of the subject.

After appropriately packaging the nasal spray composition in a squeeze bottle, for example, the nasal spray composition may be intranasally administered to one or both nasal cavities of the subject at a desired dosage. For example, the plastic dispensing tube may be appropriately placed in one nostril of the subject. The squeeze bottle may then be squeezed so that the nasal spray composition is aerosolized into a fine droplet mist and spread across the nasal mucosa of the subject. The dosage frequency of the nasal spray composition may vary depending upon personal or medical needs of the subject. Generally, dosage frequencies may range from about once per day, per nostril to about four times daily. A typical dose may contain, for example, two sprays per nostril BID.

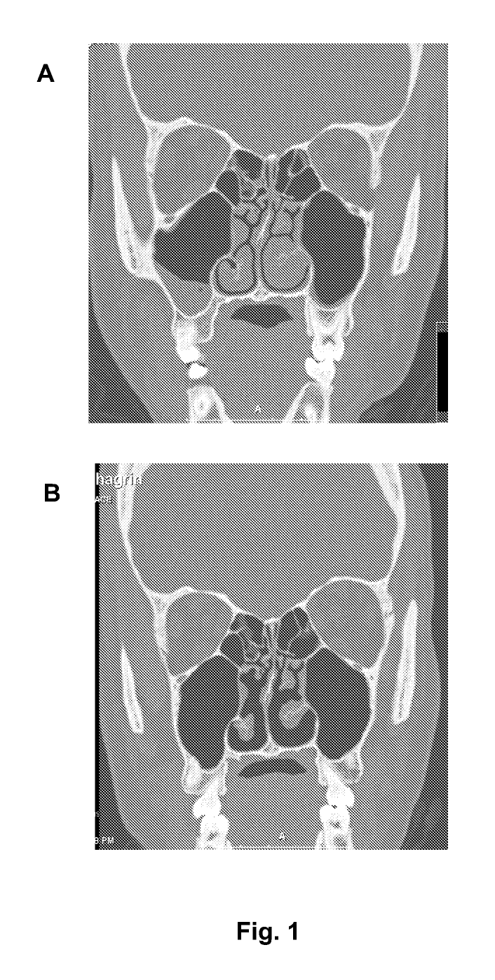

Administering the nasal spray composition may reduce or eliminate the symptoms associated with chronic sinusitis. As shown in FIG. 1B, for example, administration of the nasal spray composition can significantly reduce swelling or inflammation of the nasal turbinates. By providing a non-habituating, fast-acting, and efficacious nasal spray composition, the present invention may be useful for preventing or reducing RM while also reducing or eliminating mucosal inflammation associated with rhinitis, sinusitis, or both.

The following examples are for the purpose of illustration only and are not intended to limit the scope of the claims, which are appended hereto.

### Example 1

A NASACORT AQ/AFRIN mixture is prescribed 2 to 3 times per week, with two intranasal administrations to each nostril twice for each day. The NASACORT AQ/AFRIN mixture is made by mixing a 1 oz. bottle of AFRIN with four sample bottles (or one prescription size bottle) of NASACORT AQ. Patients are generally adults with refractory nasal symptoms, especially severe nasal congestion, who have failed multiple oral and intranasal medications, including corticosteroids. The patients typically have allergic rhinitis, vasomotor rhinitis, chronic sinusitis, or a combination of forms of rhinitis. The patients typically have symptoms interfering with work, daily activities, and/or especially sleep. Many of the patients have seen multiple physicians without relief. The response to the NASACORT AQ/AFRIN mixture is good to excellent in at least 80% of the patients. The only side effect observed has been nasal burning or stinging, which is usually not sufficient to stop the medication (except for one recent older adult woman who discontinued the mixture because of severe burning in the nasal passages).

### Example 2

E.S. is a 78 year-old man with severe vasomotor rhinitis which severely interferes with his sleep. He has been on the NASACORT AQ/AFRIN mixture for less than 1 year and is very satisfied, especially because he can now sleep through the night. He has no complications and has continued to use the medication BID or less, showing no rebound or habituation.

### Example 3

C.G. is a 56 year-old woman seen in follow-up after one month of beginning use of the NASACORT AQ/AFRIN mixture. She has allergic rhinitis, severe nasal congestion, and has failed the usual oral and intranasal medications. Her nocturnal nasal symptoms interfered with her sleep, but more importantly prevented her from using a C-PAP device for sleep apnea. On her return visit, she was not only able to sleep through the night, but was also successfully using her C-PAP device.

### Example 4

A.W. is a 42 year-old woman who was seen for a routine monthly follow-up visit. Over several months prior, she had been prescribed numerous intranasal and oral medications with insufficient relief of symptoms. She reported that after using the NASACORT AQ/AFRIN mixture for the past several months, her symptoms were well controlled. She had a normal exam, was given refills, and will be followed on a yearly basis.

From the above description of the present invention, those skilled in the art will perceive improvements, changes and modifications. For example, it will be appreciated that the dosage and concentration ranges for the decongestant and the therapeutic agent(s) comprising the nasal spray composition may vary depending upon the medical needs of the subject, the judgment of a medical professional, and/or the availability of decongestants and therapeutic agents at particular concentrations. Such improvements, changes and modifications within the skill of the art are intended to be covered by the appended claims.

Having described the invention, I claim:

1. A nasal spray composition for treating mucosal inflammation associated with rhinitis, sinusitis or both, the composition comprising:
a decongestant; and
at least one therapeutic agent selected from the group consisting of an anti-inflammatory agent and an anti-histamine agent;
wherein the nasal spray composition is non-habituating and is administered intranasally to a subject in need thereof.

2. The composition of claim 1, the nasal spray composition comprising a pharmaceutically acceptable carrier to facilitate intranasal administration of the nasal spray composition.

3. The composition of claim 1, the decongestant selected from the group consisting of oxymetazoline hydrochloride, naphazoline hydrochloride, phenylethylamine, dopamine, dobutamine, colterol, ethylnorepinephrine, isoproterenol, isoetharine, metaproterenol, terbutaline, metaraminol, tyraine, hydroxyamphetamine, ritodrine, prenalterol, methoxyamine, albuterol, amphetamine, methamphetamine, benzphetamine, mephentermine, phentermine, fenfluramine, propylhexedrine, diethylpropion, phenmetrazine, phendimetrazine, xyloepinephrine, ephedrine, pseudoephedrine, phenylephedrine, phenylpropanolamine, phenylephrine hydrochloride, xylometaxoline hydrochloride, and mixtures thereof.

4. The composition of claim 3, the decongestant comprising oxymetazoline hydrochloride.

5. The composition of claim 1, the anti-inflammatory agent selected from the group consisting of a non-steroidal anti-inflammatory drug (NSAID) and a corticosteroid.

6. The composition of claim 5, the NSAID selected from the group consisting of chromones, propionic acid derivatives, acetic acid derivatives, fenamic acid derivatives, biphenylcarboxylic acid derivatives, Cox-2 inhibitors, and oxicams.

7. The composition of claim 6, the NSAID selected from the group consisting of cromolyn sodium, menthol, acetaminophen, salicylates, salsalate, sodium salicylate, diflunisal, propionic acid derivatives, acetic acid derivatives, fenamic acid derivatives, biphenylcarboxylic acid derivatives, oxicams, fenoprofen, flurbiprofen, ibuprofen, ketoprofen, naproxen, oxaprozin, etodolac, indomethacin, ketorolac, nabumetone, sulindac, tolmetin, meclofenamate, mefenamic acid, piroxicam, bromfenac, carprofen, tiaprofenic acid, cicloprofen, diclofenac, benzydomine, and mixtures thereof.

8. The composition of claim 7, the NSAID comprising cromolyn sodium.

9. The composition of claim 5, the corticosteroid selected from the group consisting of beclamethasone dipropionate, ciclesonide, loteprednol, fluticasone propionate, mometasone furoate, flunisolide, budesonide, triamcinolone acetonide, and mixtures thereof.

10. The composition of claim 9, the corticosteroid comprising triamcinolone acetonide.

11. The composition of claim 1, the anti-histamine agent selected from the group consisting of azelastine, brompheniramine maleate, chlorpheniramine maleate, doxylamine succinate, phenindamine tartate, pheniramine maleate, promethazine maleate, pyrilamine maleate, thonzylamine hydrochloride, astemizole, azatadine, acrivastine, cetirizine, clemastine, cyclizine, carebastine, cyproheptadine, carbinoxanine, descarboethoxyloratadine, desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine, triprolidine, diphenhydramine, oxatomide, setastine, tazifyline, phenyltoloxamine, and mixtures thereof.

12. The composition of claim 11, the anti-histamine agent comprising azelastine.

13. A method for treating mucosal inflammation associated with rhinitis and/or sinusitis in a subject, the method comprising intranasally administering a therapeutically effective amount of a non-habituating nasal spray composition, the nasal spray composition comprising a decongestant and at least one therapeutic agent selected from the group consisting of an anti-inflammatory agent and an anti-histamine agent.

14. The method of claim 13, the nasal spray composition comprising a pharmaceutically acceptable carrier to facilitate intranasal administration of the nasal spray composition.

15. The method of claim 13, the decongestant selected from the group consisting of oxymetazoline hydrochloride, naphazoline hydrochloride, phenylethylamine, dopamine, dobutamine, colterol, ethylnorepinephrine, isoproterenol, isoetharine, metaproterenol, terbutaline, metaraminol, tyraine, hydroxyamphetamine, ritodrine, prenalterol, methoxyamine, albuterol, amphetamine, methamphetamine, benzphetamine, mephentermine, phentermine, fenfluramine, propylhexedrine, diethylpropion, phenmetrazine, phendimetrazine, xyloepinephrine, ephedrine, pseudoephedrine, phenylephedrine, phenylpropanolamine, phenylephrine hydrochloride, xylometaxoline hydrochloride, and mixtures thereof.

16. The method of claim 15, the decongestant comprising oxymetazoline hydrochloride.

17. The method of claim 13, the anti-inflammatory agent being selected from the group consisting of an NSAID and a corticosteroid.

18. The method of claim 17, the NSAID selected from the group consisting of chromones, propionic acid derivatives, acetic acid derivatives, fenamic acid derivatives, biphenylcarboxylic acid derivatives, Cox-2 inhibitors, and oxicams.

19. The method of claim 18, the NSAID selected from the group consisting of cromolyn sodium, menthol, acetaminophen, salicylates, salsalate, sodium salicylate, diflunisal, propionic acid derivatives, acetic acid derivatives, fenamic acid derivatives, biphenylcarboxylic acid derivatives, oxicams, fenoprofen, flurbiprofen, ibuprofen, ketoprofen, naproxen, oxaprozin, etodolac, indomethacin, ketorolac, nabumetone, sulindac, tolmetin, meclofenamate, mefenamic acid, piroxicam, bromfenac, carprofen, tiaprofenic acid, cicloprofen, diclofenac, benzydomine, and mixtures thereof.

20. The method of claim 19, the NSAID comprising cromolyn sodium.

21. The method of claim 17, the corticosteroid selected from the group consisting of beclamethasone dipropionate, ciclesonide, loteprednol, fluticasone propionate, mometasone furoate, flunisolide, budesonide, triamcinolone acetonide, and mixtures thereof.

22. The method of claim 21, the corticosteroid comprising triamcinolone acetonide.

23. The method of claim 13, the anti-histamine agent selected from the group consisting of azelastine, brompheniramine maleate, chlorpheniramine maleate, doxylamine succinate, phenindamine tartate, pheniramine maleate, promethazine maleate, pyrilamine maleate, thonzylamine hydrochloride, astemizole, azatadine, acrivastine, cetirizine, clemastine, cyclizine, carebastine, cyproheptadine, carbinoxanine, descarboethoxyloratadine, desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine, triprolidine, diphenhydramine, oxatomide, setastine, tazifyline, phenyltoloxamine, and mixtures thereof.

24. The method of claim 23, the anti-histamine agent comprising azelastine.
